(19) United States
(12) Patent Application Publication
(10) Pub. No.: US 2023/0277742 A1
(43) Pub. Date: Sep. 7, 2023

(54) HAEMOCONCENTRATION DEVICE

(71) Applicant: Brightwake Limited, Nottingham (GB)

(72) Inventor: Terence GOURLAY, Erskine (GB)

(21) Appl. No.: 18/115,216

(22) Filed: Feb. 28, 2023

Related U.S. Application Data
(60) Provisional application No. 63/314,865, filed on Feb. 28, 2022.

(51) Int. Cl. A61M 1/36

(52) U.S. Cl. CPC A61M 1/3633

(57) ABSTRACT

A haemoconcentration device comprising membrane filter having both a screen filtration and a depth filtration function.

This application claims the benefit of U.S. Provisional Patent Application Ser. No. 63/314,865 filed Feb. 28, 2022, which is hereby incorporated by reference in the entirety.

### FIELD OF THE INVENTION

The present invention relates to a device for concentrating blood, in particular to a device for use in intraoperative cell salvage and re-transfusion of blood.

### BACKGROUND TO THE INVENTION

Intraoperative cell salvage is an autologous blood transfusion technique that can avoid or reduce the need for allogenic blood transfusion during surgery. It involves the collection and reinfusion of blood spilled during surgery (‘residual’ blood). This residual blood is generally highly haemodiluted, owing to the administration of essential fluids during surgery, and if returned to the patient in its raw state may lead to excessive bleeding and patient haemodilution during the critical post-surgery recovery phase. Concentration of the blood to near-normal cell concentrations (a packed cell volume (PCV) of 33-45%) renders it suitable for re-transfusion, diminishes the bleeding risk of unprocessed blood and reduces the need for donor blood products and their associated transfusion reactions.

Hemosep® is an ultrafitration and haemoconcentration system, designed to concentrate residual blood in surgery by removing the fluid component of whole blood, the plasma, from a pooled volume of blood salvaged during, or at the end of high blood loss surgery. It is available for both human and veterinary use.

Hemosep® comprises four major components:
an intraoperative pump, suction tool and blood reservoir;
the Hemosep® bag;
the Hemosep® shaker unit; and
a blood collection bag for the collection of processed blood.

The Hemosep® bag is the active processing component of the system, that is, the haemoconcentration device, also referred to as the cell concentrator, or concentrator bag. It consists of a blood bag within which is suspended a superabsorbent material enclosed within a semi-permeable membrane. Differential filtration across the semi-permeable membrane separates the cellular components of whole blood from the fluid (plasma) component. Thus, the semi-permeable, or filter, membrane ensures efficient fluid transport from the blood bag into the superabsorber, while preventing passage of the cellular components of blood, such as red blood cells, platelets, white blood cells and clotting factors, into the superabsorber.

The semi-permeable membrane originally used in the Hemosep® bag is a polycarbonate membrane in sheet form, with discrete pores of approximately 1-2 μm in diameter making up around 20% of the total area of the membrane. The polycarbonate membrane is a ‘screen’ or ‘membrane’ filter, i.e., a filter that performs separations by retaining particles larger than its pore size on the surface of the membrane. The pore size is an absolute value representing the maximum size of any particle that would be expected to pass through the membrane.

In use of the Hemosep® ultrafitration and haemoconcentration system, haemodiluted blood is aspirated directly from a surgical site into the intra-operative blood reservoir, using the suction tool, and pumped into the Hemosep® bag. The superabsorber is pre-activated by saline and the membrane is pre-wetted by saline. Movement of cells across the membrane surface is encouraged by placing the Hemosep® bag on the Hemosep® orbital shaker unit, to agitate the blood. Plasma transferred through the membrane to the superabsorber is held in the filter membrane bag in gel form, while the concentrated blood is held in the blood bag. When the concentrated blood reaches an acceptable PCV, it may then be transferred to the blood collection bag for transfusion back to the patient.

A fluid concentration device comprising an outer bag formed of an impermeable material and an inner bag formed of a permeable material, the inner bag containing an absorbent material and being fastened to and suspended within the outer bag, is described in WO 2011/061533, the contents of which are incorporated herein in their entirety.

It is highly desirable to minimize processing times in haemoconcentration using a membrane filter. The rate-limiting factor in a process of haemoconcentration using a membrane filter is generally the time involved in the movement of cells across the membrane surface. While the Hemosep® system is highly efficient, further improving processing times in use of the system would be advantageous.

### SUMMARY OF THE INVENTION

The present inventors have now found that in a process of haemoconcentration, faster processing may be achieved using a membrane filter that has both a screen filtration function and a depth filtration function.

By ‘screen filtration function’ is meant that the membrane retains particles larger than its pore size on its surface.

By ‘depth filtration function’ is meant that the membrane filters through its depth to trap and retain particulates within its depth.

Thus, in a first aspect the present invention provides a haemoconcentration device comprising:
an outer bag formed from an impermeable material;
an inner bag formed from a filter membrane; and
an absorbent material;
the inner bag being fastened to and suspended within the outer bag; and
the absorbent material being enclosed within the inner bag;
characterized in that the membrane filter of the inner bag is a membrane filter having both a screen filtration function and a depth filtration function.

In a second aspect, the present invention provides a method for haemoconcentration to reach a desired blood packed cell volume (PCV), which method involves the steps of:
providing a haemoconcentration device comprising an outer bag formed from an impermeable material; an inner bag formed from a hydrophilic membrane filter material; and an absorbent material; the absorbent material being enclosed within the inner bag and the inner bag being fastened to, and suspended within, the outer bag; and the membrane filter of the inner bag being a membrane filter having both a screen filtration function and a depth filtration function;
introducing a primer solution into the device, to wet the membrane filter and prime the absorbent material;
introducing haemodiluted blood into the outer bag;
agitating the blood for example by placing the haemoconcentration device on an shaker unit; and
continuing said agitation until a desired PCV is reached.

By ‘desired blood packed cell volume’ is meant a PCV of around 33% to around 45%.

The terms ‘filter membrane’, ‘control membrane, ‘inner bag’ and ‘membrane’ are used interchangeably herein.

The material of the membrane used in the present invention is hydrophilic. Preferably, the material is heat-weldable.

Preferably, the membrane used in the present invention has a nominal pore size of about 3 μm. The term ‘pore size’ refers to the size of particles expected to be retained by the membrane in use; the term ‘nominal pore size’ as used herein means that around 90% of particles having a larger diameter than the given nominal pore size will be retained on or within the membrane.

The material of the semi-permeable membrane used in the present invention is most preferably hydrophilic polyethersulfone (PES).

Use of the membrane filter contemplated in the present invention gives advantages in terms of the speed of blood concentration, significantly reducing processing times compared with membrane filters previously used, for example in the Hemosep® bag.

### BRIEF DESCRIPTION OF THE DRAWINGS

FIG. 1 is an exploded view of a Hemosep® bag, illustrating the structure of an embodiment of the haemoconcentration device of the present invention.

FIG. 2 is a line drawing of the Hemosep® bag, also illustrating the structure of a haemoconcentration device in accordance with the present invention.

FIG. 3 is a scanning electron microscope (SEM) image of a polycarbonate sheet membrane as discussed herein and as used in the original Hemosep® bag.

FIG. 4 is an SEM image of a PES membrane in accordance with the present invention.

FIG. 5 is a graph illustrating the PCV levels achieved in accordance with Example 2 herein.

### DETAILED DESCRIPTION OF THE INVENTION

The Hemosep® system is a system for salvaging and recycling blood during surgery, so that it can be transfused back to the patient.

By returning all cell species, including platelets and red blood cells back to the patient, it reduces post-operative bleeding and therefore improves patient outcomes.

A number of advantages are associated with the Hemosep® system, including those set out below.

As the patient's own blood is transfused, the risks of contamination and reaction are reduced.

There is a decreased need for donor blood and associated transfusion products, leading to a reduction in donor dependency.

The system assists in the reduction of post-operative bleeding, resulting in improved patient recovery.

Maintenance of platelet population means there is preservation of normal clotting function.

A reduction in inflammatory molecules results in a reduction in post-operative complications.

The system is easy to use, with very little specialist knowledge required.

Hemosep® represents a cost-effective alternative to other cell salvage devices and/or blood transfusion.

The system is passive in nature and relies on the superabsorber to remove plasma from blood constrained within a flexible reservoir. The passage of cell species is prevented by the presence of a control membrane designed to permit the passage of the fluid component of blood, but to retain the cellular component. The concentration process is assisted by agitation of the entire concentration device by an orbital shaker mechanism. Prior to deployment the superabsorber is activated by a fluid (saline) and the control membrane is “wetted”, again by saline. The device is therefore “primed” by the introduction of a quantity of saline into the flexible reservoir bag, prior to the introduction of the blood product for processing. Generally, 100 ml of Saline is utilized to effect the priming process.

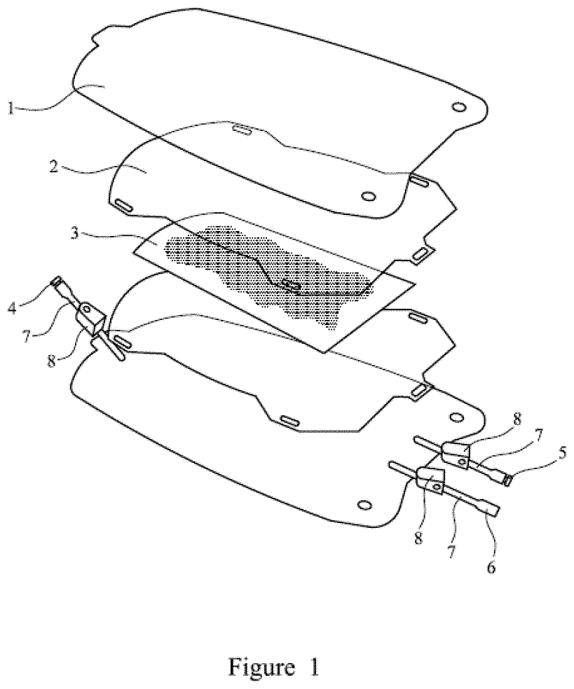

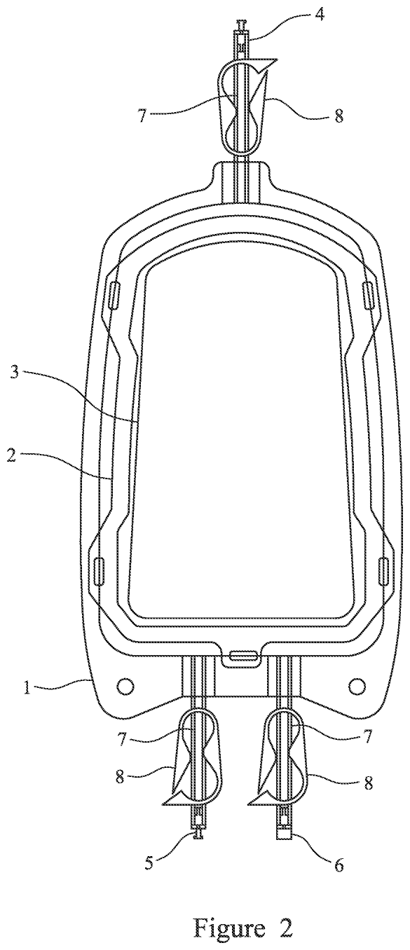

As illustrated in FIG. 1 and FIG. 2, the Hemosep® bag (the cell concentrator bag), which is the active processing section of the Hemosep® system, consists of three parts:
1. A blood bag (1) (‘outer bag’) that houses the technology (filter membrane and superabsorbent pad) and blood whilst it is filtered;
2. A filter membrane (2) (‘Membrane’/‘membrane filter’/‘filter’) that controls which components can pass into the superabsorbent pad; and
3. A superabsorbent pad (3) that absorbs unwanted blood products that pass through the filter membrane, turning it into a gel-like substance for easy disposal once complete.

As further illustrated in FIG. 1 and FIG. 2, the outer bag comprises a first inlet port (4) for the introduction of haemodiluted blood, an outlet port (5), through which processed blood may be drained (for example, into a blood collection bag) and a second inlet port (6), for the introduction of a primer fluid (for example, saline or another primer used to prime the membrane and superabsorber materials prior to use of the haemoconcentration device). Ports (4, 5, 6) are connected to the bag via tubing (for example, PVC tubing) (7). Clips or clamps (8) are provided to close off the tubes as desired.

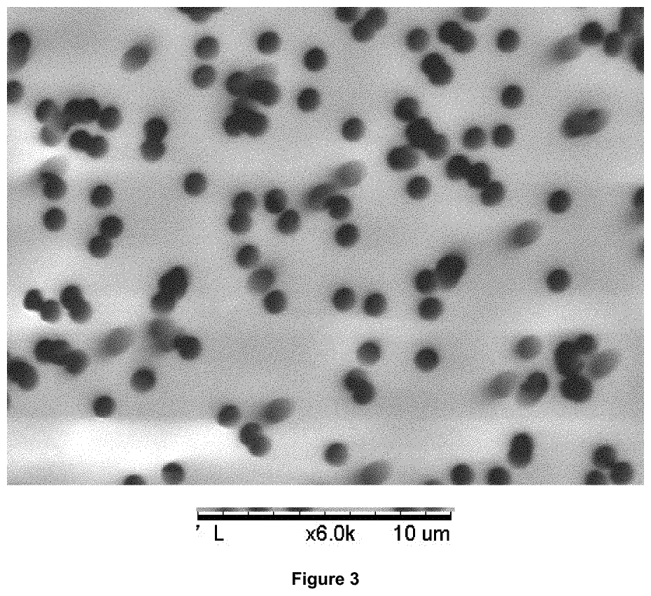

The filter membrane as originally used in the Hemosep® bag is a screening filtration membrane comprising a sheet of polycarbonate material having discrete pores with an absolute pore size of about 1-2 μm, meaning that particles of over 2 μm are retained on the surface of the membrane when in use for haemoconcentration. This membrane is illustrated in FIG. 3 herein. As can be seen from FIG. 3, the pores in the membrane take the form of ‘through-holes’, such that any particles smaller than the pore size will pass straight through the membrane, while larger particles will be unable to pass through. There is no capacity for this membrane to capture particles other than on the membrane surface. The illustrated polycarbonate sheet gives an effective open pore are of around 20%.

Generally, in use of the Hemosep® system with this polycarbonate membrane, the processing time for haemoconcentration (to reach an acceptable PCV level) is in the region of 30 minutes.

The present invention addresses the desire for shorter processing times in a haemoconcentration process such as carried out using the Hemosep® system. In accordance with the present invention, a membrane filter having both a screen filtration function and a depth filtration function is used in a haemoconcentration device such as a Hemosep® bag.

Thus, in a first aspect the present invention provides a haemoconcentration device comprising:
an outer bag (1) formed from an impermeable material;
an inner bag (2) formed from a filter membrane; and
an absorbent material (3);
wherein the absorbent material (3) is enclosed within the inner bag (2) and the inner bag (2) is fastened to, and suspended within, the outer bag (1);
characterized in that the membrane filter of the inner bag (2) is a membrane filter having both a screen filtration function and a depth filtration function.

The use of a membrane filter having both a screen filtration function and a depth filtration function gives a larger effective open pore area as compared with the polycarbonate membrane described above.

The material of the membrane used in the present invention is hydrophilic.

Preferably, the material is heat-weldable.

Preferably, the membrane used in the present invention has a nominal pore size of about 3 μm.

Most preferably, the semi-permeable membrane is formed of polyethersulfone (PES).

The haemoconcentration device of the present invention may be used in the Hemosep® system as described in the background section herein.

Thus, in use, a primer solution such as saline (for example, 100 ml saline) may be introduced into the device, for example via a first inlet port, to wet the membrane and activate the absorbent material. This may be done just prior to, for example up to 3 minutes prior to introducing blood into the device. The device may be gently agitated (for example, rocked by hand) for, for example, about 30 seconds, to distribute the saline. Haemodiluted blood may then be introduced into the device, for example via a second inlet port, to contact the membrane filter. The device may be agitated, for example by a mechanical shaker unit such as the Hemosep® shaker unit; this encourages passage of the non-cellular components of the blood across the membrane, concentrating the blood in the outer bag. Agitation may be continued for a fixed period of time to reach a PCV of 33-45%, after which the concentrated blood may be drained from the outer bag, for example by gravity and via an outlet port, into a blood collection bag, after which the device may be disposed of in a clinical waste facility. The inlet and outlet ports may be opened and closed as necessary using clips provided on the device.

Outer Bag

The outer bag houses the inner, hydrophilic membrane filter bag and the superabsorbent material and is formed of an impermeable material. Most preferably, the impermeable material of the outer bag is PVC (polyvinylchloride) in sheet form, but other synthetic plastic materials may be used, including, for example, polyethylene, polyamide, polypropylene, polyurethane, polyester, and polycarbonate, in sheet form.

Typically, the thickness of the material of the outer bag is the range of about 0.2 mm to about 3.0 mm, for example, from 0.5 mm to 2.0 mm, such as about 1.0 mm.

In use, the outer bag receives haemodiluted blood for concentration. Typically therefore, the outer bag comprises an inlet port for the introduction of haemodiluted blood. In addition, the outer bag preferably includes an outlet port, through which processed blood may be drained, for example into a blood collection bag. Saline or another primer may be used to prime the membrane and superabsorber materials prior to use of the haemoconcentration device. The outer bag may therefore include an additional inlet port, for the introduction of a primer fluid.

Preferably, the outer bag is adapted to accommodate up to about 1 litre of blood, for example, a volume of blood in the region of about 750 ml.

The outer bag may be as illustrated in FIG. 1 and FIG. 2 herein.

Absorbent Material

The absorbent material contained within the inner bag absorbs the fluid passed through the membrane filter material of which the inner bag is formed. Preferably, the absorbent material is a superabsorbent material and most preferably, a polyacrylate superabsorbent material such as, for example, cross-linked sodium poylacrylate. This is a well-known superabsorbent material. The superabsorbent material is preferably in solid sheet form and is completely enclosed within the inner bag.

Suitable superabsorbent materials other than polyacrylate materials will be known to the person skilled in the art, including, for example, polyacrylamide copolymer, ethylene maleic anhydride copolymer, cross-linked carboxymethylcellulose, polyvinylalcohol copolymers, cross-linked polyethylene oxide, starch-grafted copolymers of polyacrylonitrile, and alginates such as calcium alginate and sodium alginate.

The amount of absorbent or superabsorbent material incorporated into the device of the present invention may range from about 3 g to about 15 g.

Preferably, a superabsorbent material for use in the present invention is capable of absorbing at least about 600 ml of fluid. However, a higher capacity of up to about 3 litres of fluid may be advantageous in ensuring unidirectional flow of fluid into the inner bag, eliminating the possibility of superabsorber transferring to the outer bag.

Inner Bag—Membrane Filter

The membrane filter of the present invention has both a screen filtration function and a depth filtration function. Most preferably, it has a nominal pore size of around 3 μm.

The thickness of the membrane filter material of the inner bag is ideally in the range of 130-190 μm, in order to give optimal depth filtration function.

Preferably, the membrane filter material of the inner bag has a burst pressure of at least 0.2 bar. Burst pressure is an indication of the maximum pressure that the membrane filter is able to withstand, and a burst pressure of at least 0.2 bar is advantageous in terms of manual handling of the device, minimising the risk of rupture of the inner bag.

Preferably, the membrane used in the present invention has an air flow rate of at least 20 L/m²·s·200 Pa.

The membrane is hydrophilic.

The inner bag is preferably made by heat welding two sheets of membrane filter material together around their edges, with the superabsorbent sheet in place between the two sheets of PES. Thus, the membrane filter material is preferably heat-weldable. Other means of fastening the two sheets of material together around their edges, for example by the use of adhesives, will be known to the person skilled in the art.

Most preferably, the material of the inner bag is a polyethersulfone (PES) membrane filter.

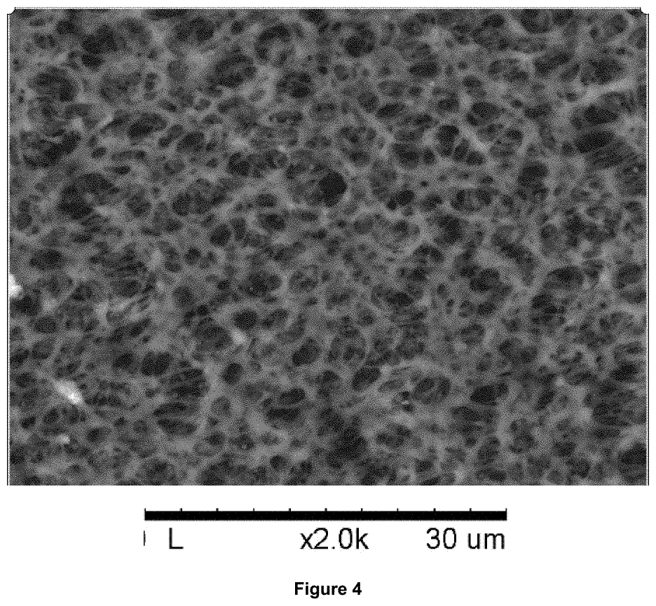

Suitable hydrophilic PES membrane filters may be sourced from Sartorius AG, Gottingen, Germany. Such a membrane is illustrated in FIG. 4 herein. As can be seen from FIG. 4, in contrast to the polycarbonate sheet membrane of FIG. 3, the illustrated PES membrane comprises a matrix of randomly oriented, bonded fibres, giving an open structure with a greater effective open area than the polycarbonate sheet membrane of FIG. 3 and providing a tortuous path through the membrane such that particles that are not captured on the surface of the membrane may be held within the bonded fibre matrix. This open, porous filter has a nominal pore size of 3 μm and retains around 90% of particles of 3 μm or above in size (diameter). A clinically insignificant percentage of smaller platelets may pass through this membrane. However, the illustrated membrane significantly outperforms the membrane of FIG. 3 when used for haemoconcentration as illustrated in the following Examples.

The Examples are based on the Hemosep® system as described herein.

Where the Examples refer to a ‘new’ membrane, control membrane or configuration, or to a ‘PES’ membrane, control membrane or configuration, the membrane is a membrane in accordance with the present invention, as follows (and as illustrated in FIG. 4):

Material Description: Polyethersulfone, hydrophilic

### Nominal Pore Size (μm): 3

### Air Flow Rate (L/m²·s·200 Pa): ≥20

### Thickness (μm): 130-180

Burst Pressure (bar): ≥0.2

Where the Examples refer to an ‘old’ or ‘original’ membrane, control membrane or configuration, or to a ‘polycarbonate’ membrane, control membrane or configuration, the membrane is a membrane of the prior art, as illustrated in FIG. 3 and described herein.

### Example 1

Blood Collection

Bovine blood was collected on the morning of the test procedures (SandyfordAbattoir Co, Sandyford Rd, Paisley, Scotland, PA3 4HP, UK), with blood taken from one animal for each batch performance test to ensure consistency of results through the avoidance of inter-animal variation. This blood was moderately haemodiluted with saline (Vetivex No 1 VO1B/3 Sodium Chloride, Henry Schein Medical, Gillingham, Kent UK) fully anti-coagulated with Heparin Sodium Salt from Porcine Intestinal Mucosa (Sigma Aldrich, Cat No: H3393-100KU, Lot #SLBN2208V).

2000 iu/500 ml of the Heparin preparation was administered to the blood at the point of collection. The blood was collected into pre-primed collection vessels which were sealed for transport to the laboratory.

Preparation of Bovine Blood for the Test Procedure

In the laboratory, the blood was gently agitated and a central sample taken for measurement of Packed Cell Volume (PCV) from each sealed blood container. This starting PCV was designated as the baseline level (BL). The target PCV (TL) for the test procedure was 20%, and this was reached by diluting the bovine blood with saline solution.

The volume of saline used to achieve the target value was calculated as follows:

Volume of Saline (ml) required to reach target level=PV(ml)×(BL/TL)%−PV(ml)
Where: Plasma Volume (PV(ml))=(100−BL)%×Blood Volume(ml)
PV=Plasma volume
BL=Baseline PCV level in % TL=Target PCV level %

The target level was set at 20% (+/−2%) for these experiments, reflecting the extreme of normal clinical blood after CPB. The acceptable range for PCV in the diluted blood was between 18.00% and 22.0%.

Conduct of the Test

The tests were carried out to ascertain the following characteristics of the Hemosep® blood cell concentration system:
(a) Concentration efficiency of a ‘new’ configuration of Hemosep® bag;
(b) Comparison with a batch of Hemosep® products with the original membrane configuration.

Fourteen (14) Hemosep® bags with a PES membrane in accordance with the present invention (‘new configuration’) were tested. A statistical power analysis confirms that with the level of consistency observed in processing over 700 laboratory performance tests, with an average SD in the region of 10% of the mean group value, a population of 14 test systems will return a statistical power of 100%. Calculations of statistical power were carried out using the DSS Researchers Toolkit (www.dssresearch.com)

The new configuration of Hemosep® devices were tested during one single laboratory session using heparinwased blood taken from a single bovine donor and stored in sealed 500 ml containers. The blood was diluted as outlined above to attain a target PVC of around 20% using saline solution. The Hemosep® bags were primed in accordance with the manufacturers IFU (instructions for use) and 500 ml of the diluted blood was introduced into the Hemosep® bags which were then placed on the Hemosep® shaker system and agitated for 40 minutes. Blood samples were taken at the start (baseline) 20 minutes and 40 minutes for measurement of Packed Cell Volume (PCV). PCV was measured manually by experienced laboratory personnel using an Adams Micro-hematochrit system (BD Adams Micro-Hematocrit II Centrifuge, Beckton, Dickenson Ltd, Oxford, UK) spinning wax sealed capillary tube samples at 11,500 RPM for 10 minutes. PCV was read from the resulting spun samples using a Hawksley 015012-00 manual hematocrit tube reader (Hawksley Ltd, Sussex, UK). This direct manual approach, although more time consuming, avoids potential inaccuracies associated with the derivation of haematocrit (PCV) values that are produced by automated systems, such as laboratory blood gas analysers. (The experienced personnel making these measurements are assessed annually to ensure that there is consistency in measurement.) All samples for trial purposes were measured in triplicate and the average used as the measured value.

Critical Measurement

The critical measurement for this process was the difference in PCV (%) over time, which represents the concentration efficiency of the system. Comparison between the original Hemosep® product and the new configuration, containing the new control membrane. A simple t-test analysis on the resultant data was applied to determine statistical significance in the difference in performance between the two configurations. A p-value of <0.05 was considered significant.

Results

### (a) Concentration Efficiency of New Configuration of Hemosep® Bags.

The increase in PCV associated with the use of the original and new configurations of Hemosep® bags in processing 500 ml of diluted blood (n=14 and n=20 respectively) are shown in Table 1.

TABLE 1
Increase in PCV associated with the new and original
configuration of Hemosep ® product (n = 14 and n = −20 respectively)

| | | | | | Change in |
|---|---|---|---|---|---|
| | | | | | PVC |
| Volume | | | 20 Minutes | 40 Minutes | over |
| Processed | | Pre-processing | Processing | Processing | processing |
| (ml) | Configuration | PCV (%) | PCV (%) | PCV (%) | period |
| 500 | New bags | 20.43 +/− 1.0220 | 38.43 +/− 1.55 | 49.14 +/− 2.03 | 28.71 +/− 1.93 |
| 500 | Original bags | 20.05 +/− 0.82 | 29.55 +/− 0.90 | 39.15 +/− 1.30 | 19.1 +/− 1.01 |

### (b) Comparison of New and Old Configuration of Hemosep® Bags

A comparison was made between data obtained from new configuration Hemosep® bags (14) and the original configuration (n=20) in processing 500 ml bovine blood using a common protocol. The result of this comparison, focusing on the specific increase in PCV levels, is shown in Table 2.

TABLE 2
Absolute change in PCV values with new and original
configurations of Hemosep ® bags (n = 14 and n = 20 respectively)

| | Change in | | Change in | |
|---|---|---|---|---|
| | PCV | | PCV | |
| Configuration | (20 mins) % | p | (40 mins) % | p |
| New | 18.0 +/− 1.41 | P = | 28.71 +/− 1.93 | P = |
| Configuration | | 7.34063E−21 | | 1.07047E−18 |
| Original | 9.5 +/− 0.84 | | 19.1 +/− 1.01 | |
| Configuration | | | | |

Analysis of Device Performance

The new configuration of Hemosep® products was clearly capable of concentrating haemodiluted blood to a greater degree over the 40 minutes processing period. The new configuration was observed to increase the PCV from a starting value of 20.43+/−1.0220 to 38.43+/−1.55 at 20 minutes and to 49.14+/−2.03 after 40 minutes of processing. This compared to a rise from 20.05+/−0.82 to 29.55+/−0.90 at 20 minutes and 39.15+/−1.30 at 40 minutes for the original configuration.

These differences were highly statistically significant at the 20 and 40 minutes processing time levels. Analysis of the data further reveals that the concentration levels observed for the new configuration after 20 minutes of processing (38.43+/−1.55) is statistically insignificantly different to the levels observed in the original configuration after 40 minutes of processing (39.15+/−1.30), p>0.05. These data confirm that the new configuration of Hemosep® bag, with the new membrane, is capable of processing the blood product to a clinically acceptable PCV level in around 50% of the time required for the original configuration. These data suggest a clinically significant reduction in processing time associated with the deployment of the new configuration.

Conclusion

The new configuration of Hemosep® bag is associated with a significant reduction in processing time required to process haemodiluted blood product to clinically acceptable levels.

### Example 2

Objectives

The objective of this study is to assess the performance of the new configuration of the Hemosep® haemoconcentration device including a PES membrane in accordance with the present invention and compare it with the original configuration.

Protocol

Two groups of Hemosep® devices (n=10 for the new membrane and n=19 for the original configuration), one group with the new membrane and one with the original configuration, were primed using 100 ml of Saline solution. The devices were then employed to process 500 ml of haemodiluted bovine blood with a packed cell volume of between 20 and 22%. As for Example 1, the blood employed for the tests was collected on the day of testing and haemodiluted using saline solution.

The anti-coagulant Heparin was introduced at the time of blood harvesting to achieve an ACT level in excess of 480 seconds. The blood was processed in the Hemosep® device for a period of 40 minutes with blood samples taken at 0, 20 and 40 minutes for the measurement of packed cell volume (PCV), the primary measure of haemoconcentration. PCV was measured manually using an Adams Micro-hematochrit system after capillary centrifugation. During the processing period the devices were agitated at a fixed cycle rate.

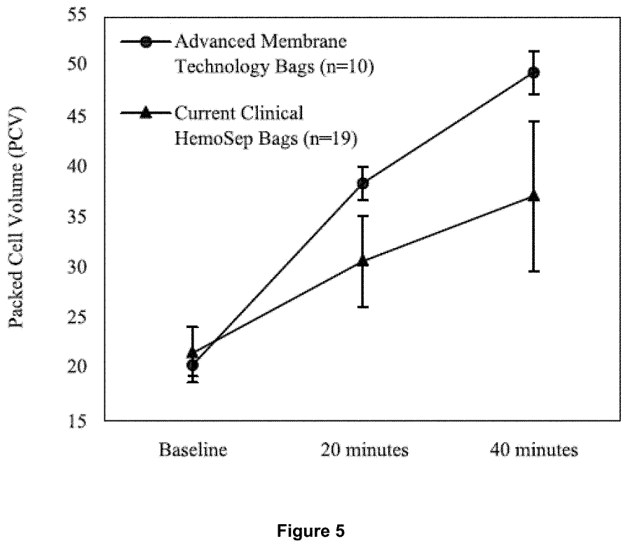

The results of this process are shown in FIG. 5 and are tabulated in Table 3. In FIG. 5, ‘Advanced Membrane Technology Bags’ refers to the device of the present invention as described and claimed herein, and ‘Current Clinical Hemosep Bags’ refers to an equivalent device containing a polycarbonate membrane in accordance with FIG. 3 herein.

TABLE 3
Performance data associated with both groups

| Process Time | | Original | |
|---|---|---|---|
| (mins) | New Membrane | Membrane | P value |
| 0 | 20.4 +/− 1.17% | 21.5 +/− 2.7% | NS |
| 20 | 38.4 +/− 1.50% | 30.65 +/− 4.42% | p <0.05 |
| 40 | 49.4 +/− 2.06% | 37.1 +/− 7.43% | P <0.05 |

Conclusion

This study has confirmed that the new membrane configuration results in an improvement in performance as reflected in the level of cell concentration over time. The difference in cell concentration achieved by the new membrane configuration was statistically significantly improved at the mid and end time-points when compared with the original membrane, resulting in an improvement in PCV in excess of 10% at the 20 and 40 minutes time-points. This improvement in haemocincentration suggests that clinically significant concentration levels, in excess of 35% can be routinely achieved after around 15 minutes with the new membrane system compared to over 30 minutes with the original configuration. Overall, these data support the use of the new membrane configuration in terms of reducing blood processing time.

The invention is further defined in the following claims.

What is claimed is:

1. A haemoconcentration device comprising:
an outer bag formed from an impermeable material;
an inner bag formed from a hydrophilic membrane filter; and
an absorbent material;
wherein the absorbent material is enclosed within the inner bag and the inner bag is fastened to, and suspended within, the outer bag;
characterized in that the membrane filter of the inner bag is a membrane filter having both a screen filtration function and a depth filtration function.

2. The haemoconcentration device of claim 1, wherein the membrane filter has a nominal pore size of about 3 μm.

3. The haemoconcentration device of claim 1, wherein the membrane filter has a thickness of about 130 μm to about 180 μm.

4. The haemoconcentration device of claim 1, wherein the membrane filter has a burst pressure of at least 0.2 bar.

5. The haemoconcentration device of claim 1, wherein the membrane filter is heat weldable.

6. The haemoconcentration device of claim 1, wherein the membrane filter has an air flow rate of at least 20 L/m²·s·200 Pa.

7. The haemoconcentration device of claim 1, wherein the membrane filter is a polyethersulfone (PES) membrane filter.

8. The haemoconcentration device of claim 1, wherein the membrane filter is a hydrophilic polyethersulfone membrane with:
a nominal pore size of 3 μm;
an air flow rate of at least 20 L/m²·s·200 Pa;
a thickness of about 130 μm to about 180 μm; and
a burst pressure of at least 0.2 bar.

9. The haemoconcentration device of claim 1, wherein the outer bag comprises an inlet port for the introduction of haemodiluted blood.

10. The haemoconcentration device of claim 1, wherein the outer bag comprises an outlet port for the drainage of processed blood.

11. The haemoconcentration device of claim 1, wherein the outer bag comprises an inlet port for the introduction of primer fluid.

12. The haemoconcentration device of claim 1, wherein the outer bag is adapted to accommodate a volume of blood of up to about 1 litre.

13. The haemoconcentration device of claim 1, wherein the outer bag is formed from polyvinylchorine (PVC).

14. The haemoconcentration device of claim 1, wherein the outer bag has a thickness of from about 0.2 mm to about 3.0 mm.

15. The haemoconcentration device of claim 1, wherein the absorbent material is a superabsorbent material.

16. The haemoconcentration device of claim 1, wherein the superabsorbent material is a polyacrylate superabsorbent material.

17. The haemoconcentration device of claim 1, wherein the superabsorbent material is in solid sheet form.

18. The haemoconcentration device of claim 1, wherein the superabsorbent material is capable of absorbing at least about 600 ml of fluid.

19. The haemoconcentration device of claim 1, wherein the amount of superabsorbent material included in the device is from about 3 g to about 15 g.

20. A method for haemoconcentration, which method involves the steps of:
providing a haemoconcentration device comprising an outer bag formed from an impermeable material; an inner bag formed from a hydrophilic membrane filter material; and an absorbent material; the absorbent material being enclosed within the inner bag and the inner bag being fastened to, and suspended within, the outer bag; and the membrane filter of the inner bag being a membrane filter having both a screen filtration function and a depth filtration function;
introducing a primer solution into the device, to wet the membrane filter and prime the absorbent material;
introducing haemodiluted blood into the outer bag;
agitating the blood for example by placing the haemoconcentration device on an shaker unit; and
continuing said agitation until a desired blood packed cell volume is reached.
